(19) United States
(12) Patent Application Publication
(10) Pub. No.: US 2007/0142495 A1
(43) Pub. Date: Jun. 21, 2007

(54) Filled polymerisable dental material and method for the production thereof

(76) Inventors: Stephan Neffgen, Hamburg (DE); Karsten Hauser, Hamburg (DE); Monika Sebald, Ritterhude (DE); Andreas Hartwig, Ritterhude (DE)

Correspondence Address: MEDLEN & CARROLL, LLP, 101 HOWARD STREET, SUITE 350, SAN FRANCISCO, CA, 94105, US

(21) Appl. No.: 10/591,744

(22) PCT Filed: Mar. 2, 2005
(86) PCT No.: PCT/EP05/02196

§ 371 Date: Sep. 1, 2006

(30) Foreign Application Priority Data
Mar. 2, 2004 (EP) 0400485.2

(51) Int. Cl. A61K 6/08

(52) U.S. Cl. 523/116; 523/223

(57) ABSTRACT

The invention relates to a filled polymerisable dental material comprising, in an organic binding agent, a partially aggregated nanometric filler and another inorganic and/or organic filler. The invention has the advantages of nanometric fillers in dental materials, based on economical aggregated fillers such as silicon dioxide obtained by pyrolysis.

The invention relates to a filled and polymerizable dental material, and to a process for the production thereof.

Various dental materials are used for prosthetic, preservative and preventive dentistry. These also include “composites”, which contain one or more filling materials in a resin matrix. The fillers impart the desired mechanical properties to the dental materials; these include the rheology in the processing phase and mechanical properties such as hardness or abrasion resistance in the cured state. It has already been proposed (WO-A-020 92 022) to improve the mechanical properties of dental materials by addition of nanoscale fillers. The nanofillers disclosed in the specification mentioned are produced by a very complicated plasma torch process (PVS).

The present invention is based on the object of creating filled and polymerizable dental materials and also a process for the production thereof, where the dental materials can be produced inexpensively and have good mechanical properties such as, for example, compressive strength and abrasion resistance.

The invention achieves this object by means of a filled and polymerizable dental material which contains:
a) an organic binder,
b) a nanoscale filler which has the following features:
   at least 50% by weight, preferably at least 60% by weight and particularly preferably at least 80% by weight of the nanoparticles have a particle diameter of less than 200 nm, preferably less than 150 or 100 nm,
   at least 20 particle number %, preferably at least 30 particle number %, preferably at least 40 particle number % and particularly preferably at least 50 particle number %, of the nanoparticles are aggregated particles,
c) at least one inorganic and/or organic filler selected from the group consisting of a ground filler having a mean particle size between 0.2 μm and 50 μm and a spherical filler having a mean particle size between 0.1 μm and 50 μm.

The process according to the invention for the production of such a dental material has the following steps:
a) making available of:
   a1) an organic binder,
   a2) an at least partially agglomerated and/or aggregated nanoscale filler,
   a3) an agent for the organic surface modification of the organic filler a2),
   a4) at least one inorganic and/or organic filler selected from the group consisting of a ground filler having a mean particle size between 0.2 μm and 50 μm and a spherical filler having a mean particle size between 0.1 μm and 50 μm;
b) carrying out an organic surface modification of the filler a2) using the agent a3);
c) incorporation of the surface-modified nanoscale filler into the organic binder until at least 50%, preferably at least 60%, further preferably at least 80%, of the nanoscale filler has a particle diameter of less than 200 nm, preferably less than 150 or 100 nm;
d) incorporation of the filler a4) into the organic binder;
   where the steps c) and d) can be carried out in any desired sequence or simultaneously and where step b) is carried out before or simultaneously to the steps c) and/or d).

The core of the invention is combination of an inexpensively obtainable nanoscale filler defined in more detail in feature b) of claim 1 with a further (micro)filler defined in more detail in feature c) of claim 1. Nanoparticles can be prepared by the sol-gel process.

In this process, alkoxysilanes are hydrolyzed and the silanols formed are condensed relatively slowly to give nanoparticles. Essentially, only “primary particles” having a very uniform size distribution result. The process is very expensive, as the particles have to be prepared completely from silanes.

The flame-pyrolytic preparation of silicic acid is likewise known. Primary particles in the nano range can likewise result here. Under the reaction conditions of flame pyrolysis, however, to a large part “agglomerates” or “aggregates” result from the primary particles. The term aggregates designates particles in which two or more primary particles are associated with one another by means of strong bonds such as, for example, sinter bridges. Agglomerates are particles in which two or more primary particles and/or aggregates are associated with one another by means of relatively weak bonds such as, for example, hydrogen bridges or dipole-dipole interactions. As a rule, in aggregates the primary particles are connected to one another superficially, in agglomerates as a rule rather point-like connections exist, i.e. the contact surfaces are comparatively small.

The aggregates and/or agglomerates formed from the primary particles are markedly larger than the primary particles, such that the flow behavior of an organic matrix containing such fillers is considerably influenced or worsened.

The invention has now recognized that, starting from fillers in which nanoscale primary particles are agglomerated and/or aggregated, it is nevertheless possible to arrive at a dental material having good mechanical properties. Preferably, these aggregated or agglomerated fillers are first organically surface-modified and subsequently incorporated into the organic binder, agglomerates and aggregates being destroyed by the incorporation to the extent that at least 50% by weight of the nanoparticles have a particle diameter of less than 100 nm (determined according to the process explained in more detail below). The invention thus allows the use of inexpensive starting materials for the nanoscale filler and nevertheless achieves that the dental materials according to the invention have the advantageous properties imparted by nanoscale fillers (in particular mechanical properties).

According to the invention, the nanoscale filler according to feature b) is still partly aggregated, i.e. a minimum amount of the nanoparticles defined in more detail in the claim are aggregated particles in which two or more primary particles and/or agglomerates are connected by strong forces. In the case of very small primary particles, the aggregates can also still have a particle diameter of less than 100 nm; frequently, however, such aggregates exceed the indicated limit of the particle diameter of 100 nm.

The invention has thus recognized that a partial aggregation of the primary particles of the nanofiller does not or only insignificantly adversely affects the advantageous properties of a nanoscale filler and that the complicated preparation of nanofillers consisting only of primary particles (for example by the sol-gel process) can be dispensed with.

Fillers such as, for example, silicon dioxide obtained by flame pyrolysis have nanoscale primary particles which are held together in relatively large aggregates and/or agglomerates both by strong aggregate forces (in particular sinter bonds) and weak agglomerate forces. The process according to the invention is based on the realization that by mechanical incorporation of such fillers into an organic binder the agglomerate bonds are largely broken and the aggregate bonds are partly broken, such that after the incorporation the filler has the parameters defined in feature b) of the claim. The organic surface modification according to feature b) of claim 16 causes fresh agglomeration of primary particles or aggregates/agglomerates after the incorporation into the organic binder to cease. This organic surface modification can in particular be a silanization explained in more detail below.

Preferably, the organic surface modification introduces groups onto the surface of the nanoscale fillers which can react chemically with the organic binder or have a high affinity for this organic binder.

The dental material according to the invention contains at least one further filler according to feature c) of claim 1. This can be a ground filler or a spherical filler (for example a spherical filler as described in DE-C 32 47 800) in each case having particle sizes defined in more detail. Both spherical and ground fillers have characteristic particle shapes, which differ markedly from the partly aggregated nano-particles according to feature b). Nanoscale primary particles can have a similar particle shape to spherical fillers according to feature c), but differ markedly in particle size.

The invention makes available a dental material which, in spite of inexpensive production, has good mechanical properties.

Advantageous embodiments of the invention are indicated in the subclaims. Claims 2 to 4 mention preferred proportions by weight of the organic binder, nanoscale filler or of the additional filler according to feature c) of claim 1.

The mean primary particle size of the nanoscale filler according to the invention is between 1 nm and 80 nm, preferably between 4 nm and 60 nm and particularly preferably between 6 nm and 50 nm. The nanoscale filler according to the invention has a BET surface area (according to DIN 66131 or DIN ISO 9277) between 15 m²/g and 600 m²/g, preferably between 30 m²/g and 500 m²/g and particularly preferably between 50 m²/g and 400 m²/g.

The nanoscale fillers employed according to the invention are preferably metal, semimetal or mixed metal oxides, silicates, nitrides, sulfates, titanates, zirconates, stannates, tungstates or a mixture of these compounds. The group consisting of the semimetals, whose properties (especially appearance and electrical conductivity) lie between those of the metals and the nonmetals, includes boron, silicon, germanium, arsenic, antimony, bismuth, selenium, tellurium and polonium (cf. Römpp Chemie Lexikon [Römpp's Chemical Encyclopaedia], Georg Thieme Verlag, 1990, p. 1711). The group consisting of the metals can be found in the periodic table left of the group consisting of the semimetals, i.e. includes the main group metals, subgroup metals, lanthanides and actinides. And the term mixed metal oxide, nitride, etc. is to be understood here as meaning a chemical compound in which at least two metals and/or semimetals together with the corresponding nonmetal anion (oxide, nitride, etc.) are combined with one another chemically.

The nanoscale fillers employed according to the invention are particularly preferably silicon dioxide, aluminum oxide, zirconium dioxide, titanium dioxide, zinc oxide, tin dioxide, cerium oxide, aluminum-silicon oxides, silicon-zinc oxides, silicon-zirconium oxides, iron oxides and their mixtures with silicon dioxide, indium oxides and their mixtures with silicon dioxide and/or tin dioxide, boron nitride, strontium sulfate, barium sulfate, strontium titanate, barium titanate, sodium zirconate, potassium zirconate, magnesium zirconate, calcium zirconate, strontium zirconate, barium zirconate, sodium tungstate, potassium tungstate, magnesium tungstate, calcium tungstate, strontium tungstate and/or barium tungstate.

In the process according to the invention, these nanoscale fillers are obtained by dispersing commercially obtainable, aggregated/agglomerated nanofillers (such as, for example, pyrogenic silicic acids) in an organic solvent and organically modifying them on the surface. During this organic modification, functional groups are applied to the surface of the nanoparticles which, on the one hand, are either covalently bonded or adsorptively bound to the nanoparticles and which, on the other hand, can react chemically with the organic binder or have a high affinity for the organic binder. In the further steps, the dispersion of the modified nanofillers in the solvent is used directly or preferably the solvent is stripped off and then the dry nanopowder is incorporated into the organic binder. The organic surface modification can also be carried out directly in the organic binder.

By means of this procedure, surprisingly the agglomerates and in some cases aggregates are permanently comminuted to the extent that at least 50%, preferably at least 60% and particularly preferably at least 80%, of the nanoparticles have a particle diameter of less than 100 nm. However, complete deaggregation is not achieved, i.e. at least 20%, preferably at least 30%, preferably at least 40% and particularly preferably at least 50%, of the nanoparticles are aggregated particles.

The organic modification of the surface of the aggregated/agglomerated nanofillers is preferably carried out by treating with a siloxane, chlorosilane, silazane, titanate, zirconate, tungstate or with an organic acid (such as are described, for example, in U.S. Pat. No. 6,387,981), an organic acid chloride or anhydride. The siloxanes, chlorosilanes, silazanes, titanates, zirconates and tungstates particularly preferably have the general formulae Si(OR′)ₙR₄₋ₙ, SiClₙR₄₋ₙ, (RₘR″₃₋ₘSi)₂NH, Ti(OR′)ₙR₄₋ₙ, Zr(OR′)ₙR₄₋ₙ and W(OR′)ₙR₆₋ₙ, where m and n is 1, 2 or 3; preferably, n is =3. The group R′ bonded via the oxygen is, just as in the case of R″, any desired organic functional group, preferably an alkyl group and particularly preferably a methyl, ethyl, propyl or isopropyl group. The functional group R is any desired organic group and is directly bonded to the silicon, titanium, zirconium or tungsten via a carbon atom. If m or n is 1 or 2, the groups R can be identical or different. R is preferably selected such that it has one or more functional groups which can react chemically with the organic binder or have a high affinity for the organic binder. These functional groups are also contained in the abovementioned organic acids, acid chlorides and anhydrides which can likewise be employed for organic surface modification. They are preferably acrylate, methacrylate, cyanoacrylate, acrylamide, methacrylamide, vinyl, allyl, epoxide, oxetane, vinyl ether, amino, acid, acid ester, acid chloride, phosphate, phosphonate, phosphite, thiol, alcohol and/or isocyanate groups. The byproducts formed in the organic surface modification of the nanofillers, such as, for example, alcohols, hydrochloric acid or ammonia, are removed in the subsequent steps except for possible residues (impurities), i.e. they are no longer contained or only contained in small amounts of ≦0.1% by weight, preferably ≦0.05% by weight, in the dental material produced later.

A further advantageous option of the invention is the organic modification of the surface of the nanofillers with dyes. In this case, the group R of the compound used for the surface modification comprises a dye or a functional group which can react with a dye. The bonding of the dye to the surface of the nanofillers can take place both by means of a covalent bond and by means of an ionic bond.

The solvent in which the organic surface modification of the nanofillers is carried out is preferably a polar aprotic solvent and particularly preferably acetone, butanone, ethyl acetate, methyl isobutyl ketone, tetrahydrofuran or diisopropyl ether. Furthermore, the direct organic surface modification of the nanofillers in the organic binder to be used for the production of the dental materials is a particularly preferred procedure. In this case, the organic binder is the solvent to be used. For the acceleration of the organic surface modification of the nanofillers, an acid can be added as a catalyst. In any case, catalytic amounts of water, preferably between 0.01% and 5%, must be present in order to carry out the modification. This water is often already present as an adsorbate on the surfaces of the aggregated/agglomerated nanofillers used as a starting substance. For assisting the reaction, further water, e.g. also in the form of a dilute acid, can be added.

In order to accelerate the disintegration of the agglomerates and aggregates in the organic solvent during the organic surface modification, before and during the modification an additional energy input can be carried out using the customary methods. This can be carried out, for example, by means of a high-speed stirrer, a dissolver, a bead mill or a mixer. In the case of the use of relatively highly viscous solvents, this is the preferred procedure, i.e. particularly if the organic binder is used directly as a solvent. If the organic binder is not used as a solvent, the organic binder to be used can be filled directly into the organic solvent with the dispersion of the organically modified nanofiller. In this case, the solvent is stripped off after the preparation of the mixture of organic binder and organically modified nanofiller or only during the later use of the nanofilled dental material. The latter is particularly a practicable procedure in the case of solvent-containing lacquers based on the dental materials according to the invention. Preferably, the organically modified nanofiller is freed of the solvent and processed further as a dry powder. In this case, the dry organically modified nanopowder is then added to the organic binder and incorporated with mechanical energy input. The incorporation can be carried out, for example, by means of a high-speed stirrer, a dissolver, a bead mill, a roll mill, a kneader or a mixer.

In the case of the use of relatively highly viscous solvents and in particular in the case of the direct use of the organic binder as a solvent, it can happen that possible excesses and/or unreacted parts of the compound used for the organic surface modification of the nanofillers cannot be removed from the dispersion. In this case, the preferred procedure is that these possible excesses and/or unreacted parts of the compound used for the organic surface modification of the nanofillers are converted by reaction with a suitable agent into substances which are then either removed from the dispersion or else can remain in the dispersion if they are inoffensive, i.e. not harmful, to humans. A particularly preferred procedure is the use of water as an agent which is reacted with possible excesses and/or unreacted parts of the compound used for the organic surface modification of the nanofillers.

The organic binder employed is a compound or a mixture of a number of compounds which contains free radical-polymerizable and/or cationically and/or anionically polymerizable groups and/or groups which allow curing by means of a condensation and/or addition reaction and/or by means of an acid-base reaction. The compounds consist of a phosphazene-based, silicon-based or organic (carbon-based) parent structure and at least one functional group, which is bonded to this parent structure and which allows curing which proceeds by means of a free radical and/or cationic and/or anionic polymerization reaction and/or by means of a condensation and/or addition reaction and/or by means of an acid-base reaction. These functional groups are preferably acrylate, methacrylate, cyanoacrylate, acrylamide, methacrylamide, vinyl, allyl, epoxide, oxetane, vinyl ether, amino, acid, acid ester, acid chloride, phosphate, phosphonate, phosphite, thiol, alcohol and/or isocyanate groups. The acid, acid ester and acid chloride groups can be derived, for example, from carboxylic acids, phosphoric acids, phosphonic acids or sulfonic acids.

The parent structure can be of linear, branched, cyclic, dendritic and/or hyperbranched construction. The parent structure can be a monomeric, oligomeric or polymeric structure. The chemical structure of the parent structure is constructed from aliphatic, cyclo-aliphatic, heterocyclic, aromatic and/or hetero-aromatic segments. One or more functional groups can be contained within the aliphatic, cycloaliphatic, heterocyclic, aromatic and/or heteroaromatic segments, e.g.

—O—, —S—, —SO—, —SO₂—, —NR¹—, —PR¹—, —P(OR¹)—, —POR¹—, —PO(OR¹)—, —O—PO(OR¹)—O—, —CO—,

—CO₂—, O—CO—O—, —COS—, —CS₂—, —C═N—, —N═C═N—, —CO(NR¹)—, O—CO—NR¹—, —NR¹—CO—NR²—, —SiR¹R²—and/or —SiR¹R²—O—, where R¹═H or any desired organic radical, preferably an unsubstituted or substituted

alkyl or aryl radical
   R²═H or any desired organic radical, preferably an unsubstituted or substituted
alkyl or aryl radical; identical to or different from R¹.

If aliphatic segments are contained in the parent structure, these are preferably derived from substituted (e.g. halogenated) and unsubstituted alkyl and alkonyl compounds, ethers, esters, carbonates, urethanes, polyethers, polyesters, polycarbonates and polyurethanes.

If cycloaliphatic segments are contained in the parent structure, these are preferably derived from substituted (e.g. halogenated) and unsubstituted cycloalkanes (such as, for example, cyclohexane and its derivatives) spirans (such as, for example, spiro-[3.3]heptane) and bi- and polycyclic hydrocarbons (such as, for example, decalin, norbornane, norcarane, pinane, adamantane, twistane and diamantane).

If heterocyclic segments are contained in the parent structure, these are preferably derived from substituted (e.g. halogenated) and unsubstituted cyclodextrins, morpholines and iminooxadiazinediones.

If aromatic segments are contained in the parent structure, these are preferably derived from substituted (e.g. halogenated) and unsubstituted benzenes (such as, for example, benzene, toluene, phenol, aniline and biphenyl) and fused aromatic ring systems (such as, for example, indene, fluorene, naphthalene, acenaphthene, anthracene, phenanthrene, naphthacene, pyrene and chrysene).

If heteroaromatic segments are contained in the parent structure, these are preferably derived from substituted (e.g. halogenated) and unsubstituted pyrroles, furans, thiophenes, indoles, benzofurans, benzothiophenes, dibenzofurans, dibenzothiophenes, pyrazoles, imidazoles, pyridines, pyrans, thiopyrans and quinolines.

As an organic binder, a liquid crystalline compound or a mixture of a number of compounds, of which at least one is liquid crystalline, can also be employed which contains free radical-polymerizable and/or cationically and/or anionically polymerizable groups and/or groups which allow curing by means of a condensation and/or addition reaction and/or by means of an acid-base reaction.

A further preferred option is the use of a fluoride ion-releasing compound or a mixture of a number of compounds, of which at least one can release fluoride ions, as an organic binder which additionally contains free radical-polymerizable and/or cationically and/or anionically polymerizable groups and/or groups which allow curing by means of a condensation and/or addition reaction and/or by means of an acid-base reaction.

As organic binders, acrylates, methacrylates, acrylamides, methacrylamides, vinyl ethers, epoxides, oxetanes, spiroorthocarbonates, spiroorthoesters, bicyclic orthoesters, bicyclic monolactones, bicyclic bislactones, cyclic carbonates, cyclic acetals, allyl sulfides, vinylcyclopropanes, organic phosphates, organic phosphonates, organic phosphites or a combination of these compounds are particularly preferably employed. Not restricting generality, some examples are mentioned below:

methyl (meth)acrylate, ethyl (meth)acrylate, n- or i-propyl (meth)acrylate, n-, i- or tert-butyl (meth)-acrylate, hexyl (meth)acrylate, 2-ethylhexyl (meth)-acrylate, cyclohexyl (meth)acrylate, isobornyl (meth)-acrylate, hydroxyethyl (meth)acrylate, hydroxypropyl (meth)acrylate, phosphoric acid esters of hydroxyethyl (meth)acrylate or hydroxypropyl (meth)acrylate, (meth)-acrylic acid, malonic acid mono(meth)acrylate ester, succinic acid mono(meth)acrylate ester, maleic acid mono(meth)acrylate ester, glyceryl (meth)acrylate, glyceryl (meth)acrylate ester, glyceryl di(meth)-acrylate, glyceryl di(meth)acrylate ester (such as, for example, glyceryl di(meth)acrylate succinate), 4-(meth)acryloyloxyethyltrimellitic acid, bis-4,6- or bis-2,5-(meth)acryloyloxyethyltrimellitic acid, 2-(((alkylamino)carbonyl)oxy)ethyl (meth)acrylate, allyl (meth)acrylate, butanediol di-meth-acrylate, hexanediol di(meth)acrylate, decanediol di(meth)acrylate, dodecanediol di(meth)acrylate, ethylene glycol di(meth)acrylate, diethylene glycol di(meth)acrylate, triethylene glycol di(meth)acrylate, tetraethylene glycol di(meth)acrylate, polyethylene glycol di (meth) acrylates, glyceryl di (meth) acrylate, glycerol propoxytri(meth)acrylate, trimethylolpropane tri-(meth)acrylate, ethoxylated and/or propoxylated trimethylolpropane tri(meth)acrylates, pentaerythritol tetra(meth)acrylate, dipentaerythritol hexa(meth)-acrylate, bisphenol A di(meth)acrylate, ethoxylated and/or propoxylated bisphenol A di(meth)acrylates, 2,2-bis-4-(3-(meth)acryloxy-2-hydroxypropoxy)phenylpropane and compounds derived therefrom, chloro- and bromo-phosphoric acid esters of bisphenol A glycidyl (meth)acrylate, urethane (meth)acrylates (such as, for example, 7,7,9-trimethylol-4,13-dioxo-3,14-dioxa-5,12-diazahexadecane 1,16-dioxydimethacrylate), polyester-urethane (meth)acrylates, polyester (meth)acrylates, polycarbonate (meth)acrylates, polyamide (meth)-acrylates, polyimide (meth)acrylates, phosphazene (meth)acrylates and siloxane (meth)acrylates; ethyl vinyl ether, n- or i-propyl vinyl ether, n-, i- or tert-butyl vinyl ether, hexyl vinyl ether, octyl vinyl ether, cyclohexyl vinyl ether, cyclohexyl 3,4-epoxy-1-methylvinyl ether, dimethanol cyclohexyl monovinyl ether, 1,4-dimethanolcyclohexyl divinyl ether, propanediol divinyl ether, butanediol divinyl ether, hexanediol divinyl ether, octanediol divinyl ether, decanediol vinyl ether, ethylene glycol divinyl ether, diethylene glycol divinyl ether, triethylene glycol divinyl ether, triethylene glycol monovinyl ether 0.30 mono(meth)acrylic acid ester, polyethylene glycol divinyl ether, tripropylene glycol divinyl ether, glyceryl trivinyl ether, pentaerythritol tetravinyl ether, 7,7,9-trimethyl-4,13-dioxo-3,14-dioxa-5,12-diazahexadecane-1,16-dioxydivinyl ether, bisphenol A divinyl ether, ethoxylated and/or propoxylated bisphenol A divinyl ether, polyester vinyl ethers, polycarbonate vinyl ethers, polyacrylate vinyl ethers, polyamide vinyl ethers, polyimide vinyl ethers, polyurethane vinyl ethers, phosphazene vinyl ethers and siloxane vinyl ethers; alkyl glycidyl ethers, glycidol, glycidyl (meth)acrylate, dipentene dioxide, 1,2-epoxy-hexadecane, bis(3,4-epoxycyclohexyl) adipate, vinyl-cyclohexene oxides, vinylcyclohexene dioxides, epoxy-cyclohexane carboxylates (such as, for example, 3,4-epoxycyclohexylmethyl 3,4-epoxycyclohexenecarboxylate), butanediol diglycidyl ether, hexanediol diglycidyl ether, dodecanediol diglycidyl ether, diglycidyl ethers of the polyethylene glycols and of the polypropylene glycols, diglycidyl ethers of substituted (e.g. halogenated) and unsubstituted bisphenols (such as, for example, bisphenol-A, bisphenol-C and bisphenol-F), resorcinol diglycidyl ether, trimethylolethane tri-glycidyl ether, trimethylolpropane triglycidyl ether, polybutadiene polyepoxides, polyester epoxides, polycarbonate epoxides, polyacrylate epoxides, polyamide epoxides, polyimide epoxides, polyurethane epoxides, phosphazene epoxides and siloxane epoxides; 3,3-disubstituted oxetanes and dioxetanes (such as, for example, 3-ethyl-3-(2-hydroxyethyl)oxetane); (trans/trans)-2,3,8,9-di-(tetramethylene)-1,5,7,11-tetraoxaspira[5.5]undecane; substituted 1,3-dioxolanes (such as, for example, 2-phenyl-4-methylene-1,3-dioxolane); difunctional 6-methylene-1,4-dithiepanes, and the reaction products of nucleophilic (meth)acrylates such as, for example, 2-hydroxyethyl (meth)acrylate or glyceryl (meth)acrylate esters with reactive phosphoric acid, phosphonic acid or phosphinic acid derivatives such as, for example, P₂O₅, POCl₃ or PCl₃.

The mixtures of organic binder and organically modified nanofiller obtained are distinguished by a high transparency and a low viscosity. In the case of use as a dental material, however, they are not suitable for all applications, since they have a high polymerization shrinkage and a relatively low mechanical strength. Therefore, according to the invention at least one inorganic and/or organic filler selected from the group consisting of ground fillers having a mean particle size between 0.2 μm and 50 μm and spherical fillers having a mean particle size between 0.1 μm and 50 μm is homogeneously incorporated into the mixtures described above. A material is thereby obtained that is distinguished by a higher compressive strength, a lower polymerization shrinkage and an improved abrasion resistance.

These dental materials according to the invention can have very different consistencies; they can be, for example, flowable and also rigid, i.e. modelable. Moreover, they can have very different rheological properties; they can be, for example, thixotropic, shear thickening or shear thinning. A high translucency, i.e. a low opacity in the dental materials according to the invention is likewise possible.

The inorganic and/or organic filler described above is incorporated into the mixture of organic binder and organically modified nanofiller with mechanical energy input. The incorporation can be carried out, for example, by means of a high-speed stirrer, a dissolver, a bead mill, a roll mill, a kneader or a mixer.

In the process according to the invention, the surface modification of the nanoscale filler is carried out either before or simultaneously to the incorporation into the organic binder. The further (spherical and/or ground) filler can be incorporated into the organic binder before, simultaneously to or after the nanoscale filler.

The inorganic and/or organic filler can be a non-reactive filler, a reactive filler or a mixture of these two types of filler. A reactive filler is understood here as meaning a filler which with ingress of water releases ions and can thus lead to a curing of the material by means of an acid-base reaction. These reactive fillers are used, for example, for the preparation of compomers and glass ionomer cements and are described, for example, in D. C. Smith, Biomaterials 19, pp. 467-478 (1998).

As an inorganic and/or organic filler, preferably quartz powder, glass powder, glass ceramic powder, metal oxides, metal hydroxides, filled and/or unfilled chip polymers, filled and/or unfilled bead polymers, spherical fillers as described, for example, in DE-C 3247800 or a mixture of these fillers are employed. The chip and bead polymers are homo- or copolymers of the polymerizable compounds (usable as an organic binder) already described, it being possible to fill these homo- or copolymers with the nanofillers and/or inorganic fillers described such as, for example, quartz powder, glass powder, glass ceramic powder, pyrogenic or wet-precipitated silicic acids. Chip polymers are obtained by grinding the corresponding polymerization products.

As an inorganic and/or organic filler, barium silicate glasses, strontium silicate glasses, borate aluminosilicate glasses, phosphate aluminosilicate glasses, fluoroaluminosilicate glasses, calcium silicates, zirconium silicates, sodium aluminum silicates, layer silicates, bentonites, zeolites including molecular sieve, the oxides and the hydroxides of the alkali metals and the alkaline earth metals, apatite, filled chip polymers, spherical fillers as described, for example, in DE-C 3247800 or a mixture of these fillers are particularly preferably employed.

A preferred variant is the use of an inorganic and/or organic filler, in which by means of organic modification functional groups are applied to the surface of the filler which can react chemically with the organic binder or have a high affinity for the organic binder. These functional groups are preferably acrylate, methacrylate, cyanoacrylate, acrylamide, methacrylamide, vinyl, allyl, epoxide, oxetane, vinyl ether, amino, acid, acid ester, acid chloride, phosphate, phosphonate, phosphite, thiol, alcohol and/or isocyanate groups. Preferably, these groups are introduced by means of the compounds already described in the surface modification of the nanofillers (siloxanes, chlorosilanes, silazanes, titanates, zirconates, tungstates, organic acids, organic acid chlorides or anhydrides).

A particularly preferred variant is the use of an inorganic and/or organic filler which is X-ray opaque and is incorporated into the dental material according to the invention in such amounts that the dental material according to the invention has an X-ray opacity (according to ISO 4049-2000) of preferably ≧100% Al and particularly preferably ≧200% Al.

For the adjustment of the viscosity of the dental material according to the invention, pyrogenic or wet-precipitated silicic acid can optionally additionally be incorporated into the dental material according to the invention.

The dental material according to the invention can be both a single-component material and a multicomponent material, where in the latter case at least one of the components, preferably all components, correspond to the composition according to the invention. The invention thus also relates to a kit for the production of a dental material according to the invention; the kit can contain one or more components. In the multicomponent kit or system, the production of a dental material according to the invention is carried out by mixing the components in the specified mixing ratio and subsequent curing.

For the curing of the dental material according to the invention an initiator or a number of initiators and optionally a coinitiator or a number of coinitiators is preferably incorporated into the dental material according to the invention. Here, initiator or initiators and coinitiator or coinitiators can be contained together in one component and/or separately in two or more components. The dental material according to the invention can thus be cured thermally, chemically, photochemically, i.e. by irradiation with UV and/or visible light, and/or by reaction with the oral and/or atmospheric moisture.

The initiators usable here can be, for example, photo-initiators. These are characterized in that they can bring about the curing of the material by absorption of light in the wavelength range from 300 nm to 700 nm, preferably from 350 nm to 600 nm and particularly preferably from 380 nm to 500 nm, and optionally by additional reaction with one or more coinitiators. Preferably, phosphine oxides, benzoin ethers, benzil ketals, acetophenones, benzophenones, thioxanthones, bisimidazoles, metallocenes, fluorenes, α-dicarbonyl compounds, aryldiazonium salts, arylsulfonium salts, aryliodonium salts, ferrocenium salts, phenylphosphonium salts or a mixture of these compounds are employed here.

Particularly preferably, diphenyl-2,4,6-trimethyl-benzoylphosphine oxide, benzoin, benzoin alkyl ethers, benzil dialkyl ketals, α-hydroxyacetophenone, dialkoxy-acetophenones, α-aminoacetophenones, i-propylthio-xanthone, camphorquinone, phenylpropanedione, 5,7-diiodo-3-butoxy-6-fluorone, (eta-6-cumene)(eta-5-cyclopentadienyl iron hexafluorophosphate, (eta-6-cumene)(eta-5-cyclopentadienyl)iron tetrafluoroborate, (eta-6-cumene)(eta-5-cyclopentadienyl)iron hexafluoro-antimonate, substituted diaryliodonium salts, triaryl-sulfonium salts or a mixture of these compounds are employed.

As coinitiators for photochemical curing, tertiary amines, borates, organic phosphites, diaryliodonium compounds, thioxanthones, xanthenes, fluorenes, fluorones, α-dicarbonyl compounds, fused polyaromatics or a mixture of these compounds are preferably employed. Particularly preferably, N,N-dimethyl-p-toluidine, N,N-dialkylalkylanilines, N,N-dihydroxy-ethyl-p-toluidine, 2-ethylhexyl p-(dimethylamino)-benzoate, butyrylcholine triphenylbutylborate or a mixture of these compounds are employed.

As initiators, “thermal initiators” can also be employed which can bring about the curing of the material by the absorption of thermal energy at elevated temperature. In this context, preferably inorganic and/or organic peroxides, inorganic and/or organic hydroperoxides, α,α′-azobis(isobutyroethyl ester), α,α′-azobis(isobutyronitrile), benzopinacols or a mixture of these compounds are employed. Particularly preferably, diacyl peroxides such as, for example, benzoyl peroxide or lauroyl peroxide, cumene hydroperoxide, benzopinacol, 2,2′-dimethylbenzopinacol or a mixture of these compounds are employed.

For chemical curing at room temperature, inter alia, a redox initiator system is used which consists of one or more initiators and a coinitiator or coinitiators serving as an activator. For reasons of the storage stability, initiator or initiators and coinitiator or coinitiators are incorporated into parts of the dental material according to the invention which are spatially separate from one another, i.e. a multicomponent preferably a two-component, material is present. As the initiator or initiators, preferably inorganic and/or organic peroxides, inorganic and/or organic hydro-peroxides, barbituric acid derivatives, malonyl-sulfamides, protic acids, Lewis or Broensted acids or compounds which release such acids, carbenium ion donors such as, for example, methyl triflate or triethyl perchlorate or a mixture of these compounds and, as a coinitiator or as coinitiators, preferably tertiary amines, heavy metal compounds, in particular compounds of the 8th and 9th group of the periodic table (“iron and copper group”), compounds having ionically bound halogens or pseudohalogens such as, for example, quaternary ammonium halides, weak Broensted acids such as, for example, alcohols and water or a mixture of these compounds are employed.

In the dental material according to the invention, any conceivable combination of the initiators and coinitiators described above can also be contained. An example of this is “dual-curing dental materials”, which contain both photoinitiators and optionally the corresponding coinitiators for photochemical curing and initiators and corresponding coinitiators for chemical curing at room temperature.

For the adjustment of specific properties, the dental material according to the invention can additionally contain “additives” or “modifiers”. Without restricting generality, some examples are mentioned below: inorganic and/or organic color pigments or dyes, stabilizers (such as, for example, substituted and unsubstituted hydroxyaromatics, tinuvins, terpinenes, phenothiazine, “HALS”—hindered amine light stabilizers—or heavy metal scavengers such as EDTA), plasticizers (such as, for example, polyethylene glycols, polypropylene glycols, unsaturated polyesters, phthalates, adipates, sebacates, phosphoric acid esters, phosphonic acid esters and/or citric acid esters), ion-emitting substances, in particular those which release fluoride ions (such as, for example, sodium fluoride, potassium fluoride, yttrium fluoride, ytterbium fluoride and/or quaternary ammonium fluorides), bactericidal or antibiotically active substances (such as, for example, chlorhexidine, pyridinium salts, penicillins, tetracyclines, chloramphenicol, antibacterial macrolides and/or polypeptide antibiotics) and/or solvents (such as, for example, water, acetone, ethanol, i-propanol, butanone and/or ethyl acetate).

The dental material according to the invention can be used for prosthetic, preservative and preventive dentistry. Without claim to completeness, some use examples may be mentioned representatively:

tooth filling materials, stump buildup materials, materials for temporary crowns and bridges, dental cements, adhesives, materials for artificial teeth, veneer materials, sealing materials and dental lacquers.

The invention is illustrated below with the aid of exemplary embodiments. First, various measurement and test procedures used are explained, subsequently the examples and comparative examples follow.

### I. Measurement and Test Procedures

1. Particle Size Determination of the Nanoscale Fillers

The particle size determinations were carried out by means of dynamic light scattering (3D-PCS). The method allows the determination of the proportions by weight of particle sizes in the range from 1 nm up to a few micrometers. The upper limit of the method is afforded in that larger particles sediment in the measurement solution and thus cannot be determined.

All samples were investigated as dilute dispersions in 2-butanone, a solids content of approximately 0.5% by weight being set. This dilution was chosen primarily in order to safely exclude particle-particle interactions.

2. Particle Size Mood of the Microfillers

The particle size distribution is determined by means of laser diffractometry (type: Coulter LS 130). In this procedure, the proportion by weight of particles which have a certain size is determined. A characteristic is the d₅₀ value, which indicates that half (50%) of the total mass of the particles exceed or fall below this size. The measurement of the particles is carried out in dilute, usually aqueous, dispersions.

3. Determination of the Particle Number Content of the Aggregates of the Nanoscale Fillers

The dispersion of filler and organic binder is investigated by means of an electron microscope. In the analysis, those particles are counted in which primary particles are bonded superficially to one another (aggregated).

4. Polymerization Shrinkage

The polymerization shrinkage was determined using the “buoyancy method”. This method operates according to the principle of the hydrostatic balance on the basis of Archimedes principle: The buoyancy which a body experiences is equal to the weight of the amount of liquid displaced by it. Thus the buoyancy is equal to the weight loss which it experiences after immersion in a liquid.

In distilled water at 4° C., the buoyancy and thus the weight loss is directly equal to the volume of the body investigated.

For the measurement of the polymerization shrinkage, i.e. the volume decrease, in the present cases the sample to be measured in each case was fixed to a very thin thread and connected firmly to the balance dish of an electronic analytical balance (Sartorius A200 S). The dry weight mₐᵢᵣ was measured. A water-filled beaker in which the sample was completely immersed was then additionally introduced into the balance. The mass of the sample m₁ and also the water temperature and thus the density of the water ρ₁ were determined. The sample was subsequently cured using an Espe Elipar II halogen lamp (three times 40 seconds) and stored dry for 24 hours at 23° C. The mass of the sample m₂ (again completely immersed in water) and the water temperature and thus the density of the water ρ₂ were determined. The polymerization shrinkage ΔV was calculated according to the following formula:
ΔV[%]=100 [Δm₁−(ρ₁/ρ₂)Δm₂]/Δm₁
In the equation: Δm₁=mₐᵢᵣ−m₁ and Δm₂=Mₐᵢᵣ−m₂
5. Flexural Strength

With the aid of a mold made of stainless steel, test bodies having the dimensions (40±2) mm×(2±0.1) mm×(2±0.1) mm were prepared (curing in a Dentacolor XS light oven: 90 seconds per side). The test bodies were stored in distilled water for 23 hours at 40° C. and then stored for 1 hour at 23° C. Subsequently, the flexural strength was measured according to ISO 4049-2000 with the aid of a universal test machine from Zwick (type Z 010/TN2A):
constant advance rate: 0.8 mm/min
calculation of the flexural strength (FS) according to the following formula:
   FS[MPa]=(3 F1)/(2 b h²)
   where
F is the maximum force exerted on the test body, in Newtons;
l is the distance between the supports with an accuracy of ±0.01 mm, in millimeters;
b is the breadth of the test body measured immediately before testing; in millimeters;
h is the height of the test body measured immediately before testing; in millimeters.
   6. Compressive Strength

With the aid of a mold made of stainless steel, cylindrical test bodies having a height of 4±0.02 mm and a diameter of 2±0.01 mm were prepared (curing using an Espe Elipar II halogen lamp: 40 seconds per side). The test bodies were stored in distilled water for 23 hours at 40° C. and then stored for 1 hour at 23° C. Subsequently, the compressive strength was measured with the aid of a universal test machine from Zwick (type Z 010/TN2A):
constant advance rate: 1.0 mm/min
calculation of the compressive strength (CS) according to the following formula:
   CS[MPa]=F/(πr²)
   where
F is the maximum force exerted on the test body, in Newtons;
r is the radius of the test body; in millimeters.
   7. Diametrical Tensile Strength

With the aid of a mold made of stainless steel, cylindrical test bodies having a height of 3±0.01 mm and a diameter of 6±0.01 mm were prepared (curing in a Dentacolor XS light oven: 90 seconds per side). The test bodies were stored in distilled water for 23 hours at 40° C. and then stored for 1 hour at 23° C. Subsequently, the diametrical tensile strength was measured according to ADA specification No. 27 of 1977 with the aid of a universal test machine from Zwick (type Z 010/TN2A)
constant advance rate: 1.0 mm/min
calculation of the diametrical tensile strength (DT) according to the following formula:
   DT[MPa]=2 F/(πdh)
   where
F is the maximum force exerted on the test body, in Newtons;
d is the diameter of the test body; in millimeters.
h is the height of the test body; in millimeters.
   8. Barcol Hardness

With the aid of a mold made of stainless steel, cylindrical test bodies having a height of 2.5±0.1 mm and a diameter of 25±0.1 mm were prepared (curing in a Dentacolor XS light oven: 180 seconds per side). Subsequently, the Barcol hardness was measured with the aid of a Barber-Colman impressor. Here, in each case at least 5 values distributed on the test body were measured and the mean value was formed.

9. Polymerization Depth

The polymerization depth was determined according to ISO 4049-1988. Here, a hollow cylinder having a height of 10 mm and a diameter of 5 mm was selected as the test body mold. The respective sample was exposed for 20 seconds using a Kulzer Translux EC halogen lamp.

10. Water Absorption

The water absorption was determined according to ISO 4049-2000. Here, a hollow cylinder of aluminum having a height of 1.0±0.1 mm and an internal diameter of 15±0.1 mm was selected as the test body mold. The respective sample was exposed for 180 seconds each side in a Dentacolor XS light oven.

11. Abrasion

The abrasion was determined using the method of 3-media abrasion developed in 1986 by DeGee (cf. A. J. DeGee et al., J. Dent. Res. 65 (5), 1986, pp. 654-658).

For the preparation of the test bodies, a mold adapted to the low spots of the sample wheel was used. The material was introduced into the mold in layers (about 2 mm per layer) and cured for 40 seconds in each case using an Espe Tri-Light halogen lamp. Immediately thereafter, the samples were additionally recured for 90 seconds in a Kuraray CS 110 light oven. The various material samples were stuck in random arrangement to the sample wheel of the 3-media abrasion machine. To guarantee a uniform rounding and surface, the sample wheel was ground flat in the wet-grinding procedure (1000 grid). Immediately after preparation, storage was carried out for at least two weeks at 37° C. in distilled water.

All materials were loaded for four times 50,000 cycles in the 3-media abrasion machine according to the described procedure (Krämer 1997). The profilometric measurement of the abrasion body was carried out by means of a computer-assisted perthometer C5D (Perthen, Göttingen). The 3-D data thus obtained were then analyzed with the aid of the image processing user interface XPERT for Windows. To this end, the abrasion range was accurately defined and output from the abraded volume of the mean height loss underneath the drawn plane.

### II EXAMPLES AND COMPARATIVE EXAMPLES

Without restriction of the generality, the invention is illustrated in more detail below with the aid of a number of examples.

Nanofiller A:

First, 150 g of Aerosil OX 50 (Degussa AG) were added to a reactor vessel and 220 g of dichloromethane were sucked into the reactor vessel in vacuo and with stirring. Afterwards, a solution of 7.6 g of 3-meth-acryloxypropyltrimethoxysilane (ABCR GmbH & Co. KG), 3.3 g of distilled water and 12 mg of methacrylic acid in 580 g of dichloromethane was likewise sucked into the vessel. After completion of this operation, the solvent mixture was distilled off in vacuo and a white powder was obtained.

Nanofiller B:

150 g of Aerosil 200 (Degussa AG) were weighed into a 2 l two-necked flask and treated with about 1000 g of 2-butanone. The initially pasty mass was stirred with a KPG stirrer until a homogeneous liquid suspension was formed. 95.55 g of 3-methacryloxypropyltrimethoxysilane were then added dropwise using a dropping funnel. The watery suspension was stirred for a total of 48 hours. Subsequently, the 2-butanone was slowly stripped off on a rotary evaporator. After the removal of the solvent, a white, fluffy, coarsely particulate porous powder remained, which easily decomposed.

Resin A:

1.97 g of camphorquinone, 3.21 g of 2-ethylhexyl 4-(dimethylamino)benzoate and 65.8 mg of 2,6-bis-tert-butyl-4-methylphenol were dissolved in 649.7 g of a 1:1 mixture of bisphenol A diglycidyl methacrylate and triethylene glycol dimethacrylate.

Volume Shrinkage after 24 h: 10.7%

Nanofilled Resin B-1 (Precursor of Reference Example 2-1):

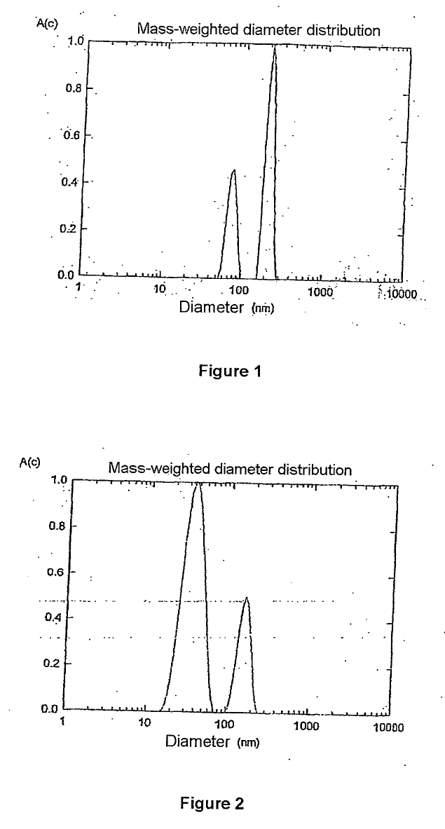

A total of 96.7 g of the nanofiller A were incorporated in portions into 100 g of resin A in the course of 2 hours with the aid of a Dispermat. In this manner, a slightly opaque, medium-viscosity, filled resin having a degree of filling of 49.2% of nanoscale silicon dioxide was obtained. The particle size distribution of the nanofilled resin B-1 ought to be measured with the aid of dynamic light scattering. The problem occurred here, however, that very large aggregates and/or agglomerates (having a mean particle size d₅₀>1 μm) were present in the resin, which separated before or during measurement and thus were unable to be additionally measured. Therefore in FIG. 1 the particle size distribution only of the particles of nanofilled resin B-1 which were able to be measured is depicted.

Transparency: 56.2%

Volume shrinkage after 24 h: 5.2%

FIG. 1 shows the particle size distribution of the unseparated particles of the nanofilled resin B-1(dynamic light scattering)

Peak 1:

Mean value=75.8 nm; breadth=12.5%; amplitude=

34.5%

Peak 2:

Mean value=212.7 nm; breadth=11.1%; amplitude=65.5%

Taking into consideration the relatively high proportion of unmeasurable aggregated and/or agglomerated particles having a particle size d>1 μm, it can be concluded from the above figure that only a very small proportion of the particles of the nanofilled resin

B-1 has a particle size d<100 nm.

Nanofilled Resin B-2 (Precursor of Reference Example 2-2):

A total of 96.7 g of the nanofiller A were incorporated in portions into 100 g of the resin A within one hour with the aid of a laboratory mixer. In this manner, a slightly opaque, medium-viscosity, filled resin having a degree of filling of 49.2% of nanoscale silicon dioxide was obtained.

In the case of this resin, it was not possible to measure the particle size distribution with the aid of dynamic light scattering, since at least 80% of the filler was so strongly aggregated and/or agglomerated that the corresponding particles were so large that they separated and were thus unable to be measured.

Transparency: 56.4%

Volume shrinkage after 24 h: 5.3%

Nanofilled Resin C (Precursor of Inventive Examples):

Approximately 50 g of the nanofiller B were added to 250 g of a 1:1 mixture of bisphenol A diglycidyl methacrylate and triethylene glycol dimethacrylate and incorporated in 90 minutes at 1200 rpm with the aid of a Dispermat. A further 40 g of the nanofiller B were then added, and the mixture was dispersed for 1 hour at 1000 rpm and subsequently overnight at 500 rpm. A total of 225 g of the nanofiller were dispersed in portions of 30-40 g. Subsequently, 0.16% by weight of camphor-quinone, 0.26% by weight of 2-ethylhexyl 4-(dimethyl-amino)benzoate and 0.005% by weight of 2,6-bis-tert-butyl-4-methylphenol were dissolved in this homogeneous mixture. In this manner, a very transparent, medium-viscosity, filled resin having a degree of filling of 47.2% of nanoscale silicon dioxide was obtained.

In contrast to the nanofilled resins B-1 and B-2, no separation occurred in the case of this resin, so that in this case it was possible to determine the particle size distribution of all particles by means of dynamic light scattering (see FIG. 2).

Transparency: 94.5%

Volume shrinkage after 24 h: 7.3%

FIG. 2 shows a particle size distribution of nanofilled resin C (dynamic light scattering)

Peak 1:

Mean value=35.2 nm; breadth=31.9%; amplitude=77.2%

Peak 2:

Mean value=159.1 nm; breadth=17.5%; amplitude=22.8%

The above figure clearly proves that more than 50% of the particles of the nanofilled resin C have a particle

size d<100 nm.

In the following examples 1-4, hybrid composites are presented which were prepared according to the prior art (reference examples) or according to the present invention. It is to be noted that all 5 pastes were adjusted sensorially to the same consistency.

### Example 1 (Reference)

6.0 g of Aerosil R 974 (Degussa AG) and 130.7 g of silanized barium silicate glass having a mean particle size of 1.0 μm were incorporated into 34.1 g of resin A. Subsequently, the mixture was degassed at about 200 mbar for about 60 minutes. In this manner, a solid, modelable, light-curing paste was obtained.

Composition: See Table 1

Properties: See Table 2

### Example 2-1 (Reference)

70.6 g of silanized barium silicate glass having a mean particle size of 1.0 μm were initially incorporated into 50.0 g of the nanofilled resin B-1. The resulting paste, however, was so solid and crumbly that it was additionally diluted with 10.0 g of resin A. Subsequently, the paste was degassed at about 200 mbar for about 60 minutes. In this manner, a solid, modelable, light-curing paste was obtained.

Composition: see Table 1

Properties: see Table 2

### Example 2-2 (Reference)

60.8 g of silanized barium silicate glass having a mean particle size of 1.0 μm were initially incorporated into 50.0 g of the nanofilled resin B-2. The resulting paste, however, was slightly too solid and was therefore additionally diluted with 1.0 g of resin A. Subsequently, the paste was degassed at about 200 mbar for about 60 minutes. In this manner, a solid, modelable, light-curing paste was obtained.

Composition: see Table 1

Properties: see Table 2

### Example 3 (Invention)

105.5 g of silanized barium silicate glass having a mean particle size of 1.0 μm were incorporated into 50.1 g of the nanofilled resin C. Subsequently, the paste was degassed at about 300 mbar for about 60 minutes. In this manner, a solid, modelable, light-curing paste was obtained.

Composition: See Table 1

Properties: See Table 2

### Example 4 (Invention)

6.0 g of Aerosil R 974 (Degussa AG) and 95.6 g of silanized barium silicate glass having a mean particle size of 1.0 μm were incorporated into 54.1 g of the filled resin C. Subsequently, the paste was degassed at about 200 mbar for about 60 minutes. In this manner, a solid, modelable, light-curing paste was obtained.

Composition: See Table 1

Properties: See Table 2

TABLE 1
Percentage composition of the hybrid
composites (Exs. 1-4)

| | Ex. 1 | Ex. 2-1 | Ex. 2-2 | Ex. 3 | Ex. 4 |
|---|---|---|---|---|---|
| | (reference) | (reference) | (reference) | (invention) | (invention) |
| Resin A | 20.0% | 7.65% | 0.9% | — | — |
| Nanofilled resin B-1 | — | 38.3% | — | — | — |
| Nanofilled resin B-2 | — | — | 44.7% | — | — |
| Nanofilled resin C | — | — | — | 32.2% | 34.7% |
| Aerosil R 974 | 3.5% | — | — | — | 3.9% |
| Barium silicate | 76.5% | 54.05% | 54.4% | 67.8% | 61.4% |
| glass (d₅₀ = 1.0 μm) | | | | | |
| Resin A | 20.0% | 27.1% | 23.6% | 17.0% | 18.3% |
| Nanofiller A | — | 18.8% | 22.0% | — | — |
| Nanofiller B | — | — | — | 15.2% | 16.4% |
| Aerosil R 974 | 3.5% | — | — | — | 3.9% |
| Barium silicate | 76.5% | 54.1% | 54.4% | 67.8% | 61.4% |
| glass (d₅₀ = 1.0 μm) | | | | | |

TABLE 2
Physical and mechanical properties of
the hybrid composites (Exs. 1-4)

| | Ex. 1 | Ex. 2-1 | Ex. 2-2 | Ex. 3 | Ex. 4 |
|---|---|---|---|---|---|
| | (reference) | (reference) | (reference) | (invention) | (invention) |
| Compressive strength | 409 | 431 | 459 | 513 | 493 |
| [MPa] | | | | | |
| Volume shrinkage | 3.8 | 4.0 | 4.4 | 3.3 | 3.4 |
| after 24 h [%] | | | | | |
| Abrasion [μm] | 41 | n.d. | n.d. | n.d. | 20 |
| Flexural strength | 126 | 131 | 121 | 129 | 125 |
| [MPa] | | | | | |
| Diametrical tensile | 53 | 59 | 58 | 57 | 57 |
| strength [MPa] | | | | | |
| Barcol hardness | 82 | 82 | 80 | 85 | 79 |
| Polymerization | ≧10 | ≧10 | ≧10 | ≧10 | ≧10 |
| depth [mm] | | | | | |
| Water absorption | 29 | 22 | 22 | 20 | 22 |
| [μg/mm³] | | | | | |

In the following examples 5 and 6, microfiller composites are presented which were prepared according to the prior art (reference example) or according to the present invention. It is to be noted that the two pastes were adjusted sensorially to the same consistency.

### Example 5 (Reference)

14.0 g of Aerosil R 974 (Degussa AG) and 86.1 g of a ground chip polymer which contained dodecanediol dimethacrylate, trimethylolpropane trimethacrylate and silanized Aerosil OX 50 were incorporated into 40.0 g of resin A. Subsequently, the paste was degassed at about 300 mbar for about 45 minutes. In this manner, a solid, modelable, light-curing paste was obtained.

Composition: See Table 3

Properties: See Table 4

### Example 6 (Invention)

100.7 g of the ground chip polymer described in ex. 5 were incorporated into 54.0 g of the nanofilled resin C. Since the resulting paste was too solid, it was diluted with 12.4 g of the nanofilled resin C. Subsequently, the paste was degassed at about 200 mbar for about 60 minutes. In this manner, a solid, modelable, light-curing paste was obtained.

Composition: See Table 3

Properties: See Table 4

TABLE 3
Percentage composition of the microfiller
composites (Exs. 5-6)

| | Ex. 5 | Ex. 6 |
|---|---|---|
| | (reference) | (invention) |
| Resin A | 28.6% | — |
| Nanofilled resin C | — | 39.7% |
| Aerosil R 974 | 10.0% | — |
| Org. filler | 61.4% | 60.3% |
| (ground prepolymer) | | |
| Resin A | 28.6% | 21.0% |
| Nanofiller B | — | 18.7% |
| Aerosil R 974 | 10.0% | — |
| Org. filler | 61.4% | 60.3% |
| (ground prepolymer) | | |

TABLE 4
Physical and mechanical properties of
the microfiller composites (Exs. 5-6)

| | Ex. 5 | Ex. 6 |
|---|---|---|
| | (reference) | (invention) |
| Compressive strength [MPa] | 249 | 345 |

These results show that the use of the composition employed according to the invention in dental materials leads to an increase in the compressive strength, to a lowering of the polymerization shrinkage and to an improvement of the abrasion resistance.

1. A filled and polymerizable dental material, characterized in that it contains:
a) an organic binder,
b) a nanoscale filler which has the following features:
at least 50% by weight, preferably at least 60% by weight and particularly preferably at least 80% by weight of the nanoparticles have a particle diameter of less than 200 nm, preferably less than 150 nm, particularly preferably less than 100 nm,
at least 20 particle number %, preferably at least 30 particle number %, preferably at least 40 particle number % and particularly preferably at least 50 particle number %, of the nanoparticles are aggregated particles,
the nanoscale filler is organically surface-modified,
c) at least one inorganic and/or organic filler selected from the group consisting of a ground filler having a mean particle size between 0.2 μm and 50 μm and a spherical filler having a mean particle size between 0.1 μm and 50 μm.

2. The dental material as claimed in claim 1, characterized in that it contains 1 to 99% by weight, preferably 5 to 90% by weight and particularly preferably 10 to 80% by weight, of organic binder a).

3. The dental material as claimed in claim 1 or 2, characterized in that it contains 0.1 to 90% by weight, preferably 1 to 80% by weight and particularly preferably 10 to 60% by weight, of nanoscale filler b).

4. The dental material as claimed in one of claims 1 to 3, characterized in that it contains 0.1 to 95% by weight, preferably 1 to 90% by weight and particularly preferably 10 to 80% by weight, of inorganic and/or organic filler c).

5. The dental material as claimed in one of claims 1 to 4, characterized in that it additionally contains pyrogenic and/or wet-precipitated silicic acids for adjustment of the viscosity.

6. The dental material as claimed in claim 5, characterized in that it contains 0 to 30% by weight, preferably 0 to 20% by weight and particularly preferably 0 to 10% by weight of the pyrogenic and/or wet-precipitated silicic acids for adjustment of the viscosity.

7. The dental material as claimed in one of claims 1 to 6, characterized in that the organic binder a) is a compound or a mixture of a number of compounds which contains free-radical polymerizable and/or cationically and/or anionically polymerizable groups and/or groups which allow curing by means of a condensation, addition and/or acid-base reaction.

8. The dental material as claimed in one of claims 1 to 7, characterized in that the nanoscale filler b) is a metal, semimetal or mixed metal oxide, silicate, nitride, sulfate, titanate, zirconate, stannate, tungstate or a mixture of these compounds.

9. The dental material as claimed in claim 8, characterized in that the nanoscale filler b) is silicon dioxide.

10. The dental material as claimed in one of claims 1 to 9, characterized in that the filler c) is a spherical filler, quartz powder, glass powder, glass ceramic powder or a mixture of these powders.

11. The dental material as claimed in one of claims 1 to 9, characterized in that the inorganic and/or organic filler c) is a filled or unfilled chip polymer and/or bead polymer.

12. The dental material as claimed in one of claims 1 to 11, characterized in that the inorganic and/or organic filler c) is surface-modified and has functional groups on its surface which can react chemically with the organic binder a) or have a high affinity for the organic binder a).

13. The dental material as claimed in one of claims 1 to 12, characterized in that it additionally contains an initiator or a number of initiators and optionally a coinitiator or a number of coinitiators.

14. The dental material as claimed in one of claims 1 to 13, characterized in that it is X-ray opaque.

15. The use of the dental material as claimed in one of claims 1 to 14 as a material for prosthetic, preservative and preventive dentistry such as, for example, as a tooth filling material, stump buildup material, material for temporary crowns and bridges, dental cement, adhesive, material for artificial teeth, veneer material, sealing material and dental lacquer.

16. A process for the production of dental materials, having the steps:
a) making available of:
a1) an organic binder,
a2) an: at least partially agglomerated and/or aggregated nanoscale filler,
a3) an agent for the organic surface modification of the filler a2),
a4) at least one inorganic and/or organic filler selected from the group consisting of a ground filler having a mean particle size between 0.2 μm and 50 μm and a spherical filler having a mean particle size between 0.1 μm and 50 μm;
b) carrying out an organic surface modification of the filler a2) using the agent a3);
c) incorporation of the surface-modified nanoscale filler into the organic binder until at least 50% by weight, preferably at least 60% by weight, further preferably at least 80% by weight, of the nanoscale filler has a particle diameter of less than 200 nm, preferably less than 150 nm, particularly preferably less than 1.00 nm;
d) incorporation of the filler a4) into the organic binder;
where the steps c) and d) can be carried out in any desired sequence or simultaneously and where step b) is carried out before or simultaneously to the steps c) and/or d).

17. The process as claimed in claim 16, characterized in that the organic surface modification of the nanoscale fillers a2) is carried out directly in the organic binder.

18. The process as claimed in claim 16 or 17, characterized in that in step b) additional mechanical energy is introduced, preferably by means of a high-speed stirrer, a dissolver, a bead mill or a mixer.

19. The process as claimed in one of claims 16 to 18, characterized in that the organic binder a1) is a compound or a mixture of a number of compounds which contains free radical-polymerizable and/or cationically and/or anionically polymerizable groups and/or groups which allow curing by means of a condensation, addition and/or acid-base reaction.

20. The process as claimed in one of claims 16 to 19, characterized in that the nanoscale filler a2) is a metal, semimetal or mixed metal oxide, silicate, nitride, sulfate, titanate, zirconate, stannate, tungstate or a mixture of these compounds.

21. The process as claimed in claim 20, characterized in that the nanoscale filler a2) is silicon dioxide.

22. The process as claimed in one of claims 16 to 21, characterized in that in the organic surface modification groups are introduced onto the surface of the nanoscale fillers a2) which can react chemically with the organic binder a1) or have a high affinity for the organic binder.

23. The process as claimed in one of claims 16 to 22, characterized in that the agent employed for the organic surface modification is a silane, chlorosilane, silazane, titanate, zirconate and/or tungstate.

24. The process as claimed in one of claims 16 to 23, characterized in that the inorganic and/or organic filler a4) is a spherical filler, quartz powder, glass powder, glass ceramic powder or a mixture of these powders.

25. The process as claimed in one of claims 16 to 23, characterized in that the inorganic and/or organic filler a4) is a filled or unfilled chip polymer and/or bead polymer.

26. The process as claimed in one of claims 16 to 25, characterized in that the inorganic and/or organic filler a4) is organically surface-modified and thus has functional groups on its surface which can react chemically with the organic binder a1) or have a high affinity for the organic binder.
